(19) United States
(12) Patent Application Publication
(10) Pub. No.: US 2015/0050657 A1
(43) Pub. Date: Feb. 19, 2015

(54) TARGETED ENRICHMENT AND AMPLIFICATION OF NUCLEIC ACIDS ON A SUPPORT

(71) Applicant: Illumina Cambridge Limited, Nr Saffron Walden (GB)

(72) Inventors: Roberto Rigatti, Nr Saffron Walden (GB); Jonathan Boutell, Nr Saffron Walden (GB)

(73) Assignee: Illumina Cambridge Limited, Nr Saffron Walden (GB)

(21) Appl. No.: 14/377,104

(22) PCT Filed: May 28, 2013
(86) PCT No.: PCT/EP2013/052337

§ 371 Date: Aug. 6, 2014

Related U.S. Application Data
(60) Provisional application No. 61/595,983, filed on Feb. 7, 2012.

(51) Int. Cl. C12P 19/34

(52) U.S. Cl. CPC C12P 19/34
USPC 435/6.12; 435/91.2

(57) ABSTRACT

Provided herein is a method of selecting target nucleic acids on a support. The method includes providing a plurality of beads each bead comprising one or more oligonucleotides, providing a support with a plurality of primers with a sequence complementary to at least a portion of the oligonucleotides on the beads, contacting the beads with the support wherein the oligonucleotides on the beads bind to the primers on the support, performing an extension reaction by extending the primers on the support to produce capture oligonucleotides, contacting the support comprising the capture oligonucleotides with the target nucleic acids, and extending the capture oligonucleotides bound to target nucleic acids to produce target extension products comprising a sequence complementary to at least a portion of the target nucleic acids. Optionally, the method further includes amplifying the target extension products.

### BACKGROUND

With the advent of next-generation sequencing technologies it is now feasible to sequence an entire genome quickly and cheaply. For many applications though, sequencing an entire genome may not be necessary. In many instances it can be sufficient to sequence only a small fraction of the genome. This fraction could be for example represented by the exome. Whole exome sequencing has been successfully used to identify the causal mutations of many rare mendelian diseases (Ng et al., Nat. Genet. 42(1):30-5 (2010); and Ng et al., Nat. Genet. 42-(9):790-3 (2010), which are incorporated by reference herein in their entireties). In other cases, the genomic fraction of interest could be represented by a locus that has been linked to an important trait (i.e. disease susceptibility) through GWAS (genome-wide association studies). In this case, sequencing this relatively small genomic region from many individuals (with and without the disease) would be very informative and an approach involving targeted resequencing would be much cheaper and quicker than whole human genome sequencing.

In current protocols, an enrichment step to select the desired region or regions is required prior to sequencing. Enrichment can be achieved in several different ways. One way is to amplify the target sequence or sequences by PCR. Another method is to use a hybridization step to selectively capture the targets of interest. Hybridization can be done either in solution or on a solid support. The captured molecules then have to be retrieved, seeded and amplified in order to be able to sequence them. Thus, there is a need for a simplified method of selecting target nucleic acids for amplification, sequencing or other techniques.

### SUMMARY

Provided herein is a method of selecting target nucleic acids on a support. The method includes the steps of providing a plurality of beads each bead comprising one or more oligonucleotides, providing a support with a plurality of primers with a sequence complementary to at least a portion of the oligonucleotides on the beads, contacting the beads with the support under conditions wherein the oligonucleotides on the beads bind to the primers on the support, performing an extension reaction by extending the primers on the support to produce capture oligonucleotides comprising a sequence complementary to at least a portion of the oligonucleotides on the beads, contacting the support comprising the capture oligonucleotides with the target nucleic acids, the target nucleic acids potentially comprising sequences complementary to one or more of the capture oligonucleotides, and extending the capture oligonucleotides bound to target nucleic acids to produce target extension products comprising a sequence complementary to at least a portion of the target nucleic acids. Optionally, the method further includes amplifying the target extension products.

The details of one or more embodiments are set forth in the accompanying drawings and the description below. Other features, objects, and advantages will be apparent from the description and drawings, and from the claims.

### DESCRIPTION OF DRAWINGS

FIGS. 1A-1H are a schematic of an exemplary method provided herein. FIG. 1A is a schematic of target nucleic acid preparation. Briefly, nucleic acid is fragmented followed by end repair of the fragments and ligation of adaptors to the fragments. FIG. 1B shows beads coated with oligonucleotides, each bead comprising a distinct oligonucleotide specific for a certain target nucleic acid. FIG. 1C shows a support surface (e.g., flow cell surface) coated with primers complementary to a portion of the oligonucleotides on the beads. FIG. 1D shows oligo coated beads binding or hybridizing to primers on the surface of the support. FIG. 1E shows the support with primers and extension products (or capture oligonucleotides) produced by copying the oligonucleotides on the beads bound to the primers. FIG. 1F shows contacting the target nucleic acids with the support and binding of specific target nucleic acids to the capture oligonucleotides. FIG. 1G shows removal of unbound nucleic acids. FIG. 1H shows the target extension products produced by copying the captured target nucleic acids by extending the capture oligonucleotides.

FIGS. 2A-2F are a schematic of an exemplary method provided herein. FIG. 2A shows an oligonucleotide coated bead with a pair of capture target sequences specific for two regions of a target nucleic acid. FIG. 2B shows a support surface (e.g., flow cell surface) coated with primers complementary to a portion of the oligonucleotides on the beads and binding of the beads to the surface of a support via the oligos. FIG. 2C shows the support with the primers and capture oligonucleotides produced by copying the oligonucleotides hybridized to the primers. FIG. 2D shows a target nucleic acid bound to one of the capture oligonucleotides. FIG. 2E shows the target extension product produced by copying the captured target nucleic acid. FIG. 2F shows the beginning of amplification of the target extension product (or captured target nucleic acid) using the capture oligonucleotides, which serve as forward and reverse primers.

### DETAILED DESCRIPTION

Provided herein are methods for selecting target nucleic acids that simplify workflow and reduce the hands-on time required to perform experiments. With the method described herein, the step of capturing target nucleic acids allows for amplification of the target nucleic acids and to sequencing of the target nucleic acids all on the same support (e.g., customized flow cell).

Provided herein is a method of selecting and, optionally, amplifying target nucleic acids on a support. The method includes the steps of providing a plurality of beads each bead comprising one or more oligonucleotides, providing a support with a plurality of primers with a sequence complementary to at least a portion of the oligonucleotides on the beads, contacting the beads with the support under conditions wherein the oligonucleotides on the beads bind to the primers on the support, performing an extension reaction by extending the primers on the support to produce extension products comprising a sequence complementary to at least a portion of the oligonucleotides, contacting the support comprising the extension products with the target nucleic acids, the target nucleic acids potentially comprising sequences complementary to one or more of the extension products, extending the extension products bound to target nucleic acids to produce target extension products comprising a sequence complementary to at least a portion of the target nucleic acids. The method may further include amplifying the target extension products.

Generally, the capture oligonucleotides will include a region of the same sequence as the plurality of amplification oligonucleotides (i.e., primers). Once the bead comprising the oligonucleotides has hybridized to one of the amplification oligonucleotides (i.e., primers) and been extended, the bases in the capture oligonucleotide sequence will have been copied. Thus, the capture oligonucleotide may include both the amplification oligonucleotide sequence, plus a further sequence that is complementary to a region of a target nucleic acid. In one embodiment, a plurality of three types of oligonucleotides (for example comprising capture oligonucleotides, forward and reverse amplification oligonucleotides) are immobilised to a solid support.

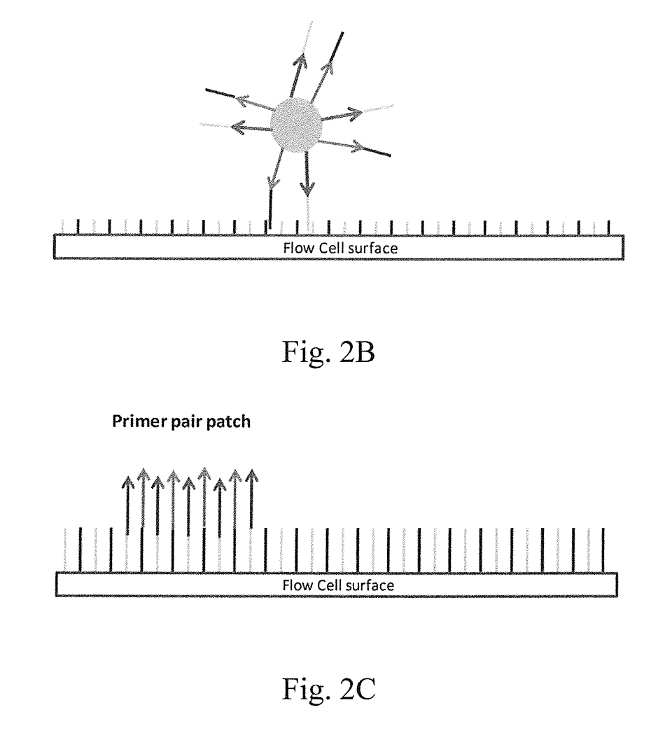

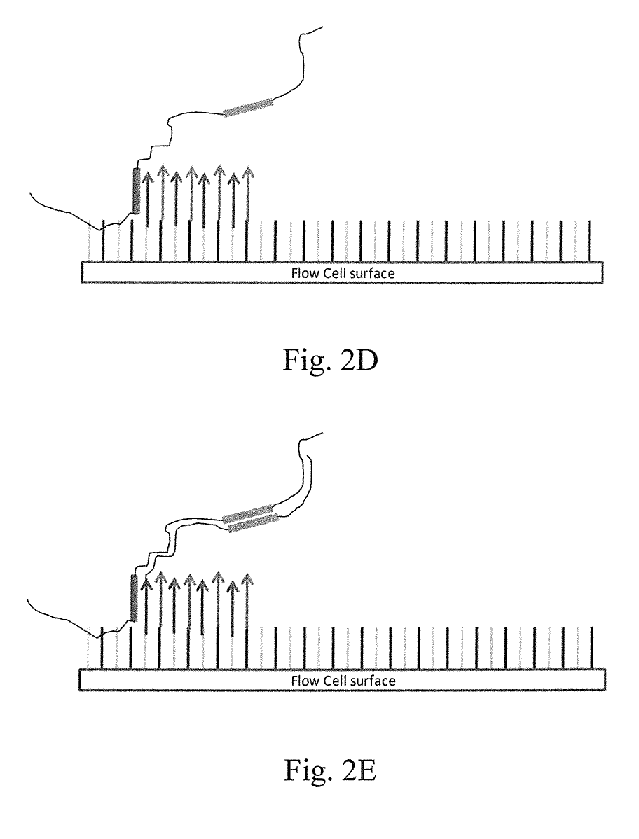

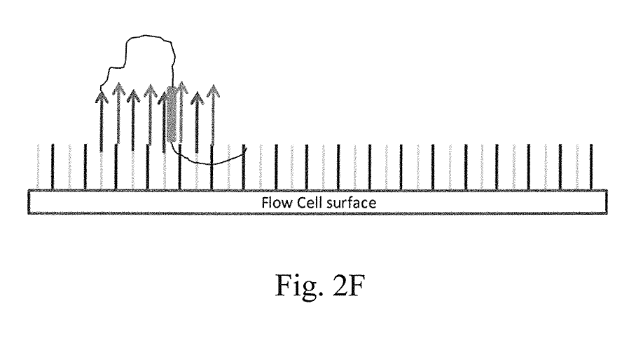

Optionally, each bead comprises a different oligonucleotide sequence. The plurality of beads can comprise two or more subsets of beads. Optionally, each bead in the subset of beads comprises the same oligonucleotide sequence. Optionally, each subset of beads comprises different oligonucleotide sequences. Optionally, each bead comprises one or more pairs of oligonucleotides comprising forward and reverse primer sequences specific to a target nucleic acid sequence (e.g., first and second oligonucleotides comprising forward and reverse primer sequences, respectively). In this embodiment, the extension products or capture oligonucleotides comprise forward and reverse primer sequences (e.g., forward and reverse capture oligonucleotides) for amplifying the target nucleic acid. The target nucleic acid to be captured can, for example, hybridize to the one of the primer sequences of the forward or reverse capture oligonucleotides and the target extension products can be amplified using the forward and reverse primer sequences of the capture oligonucleotides. By way of a further example, a pair of primers (or extension products) in each oligonucleotide patch can be generated, where half the primers are “forward” primers and half the primers are “reverse” primers to a particular genomic region of interest (FIG. 2C). FIG. 2A shows an example of such a pair of oligonucleotide primers on a bead. FIG. 2B then shows how the oligonucleotides on such a bead can be used to create an oligonucleotide patch containing a pair of primers on the surface of a support (e.g., flowcell). FIG. 2D then demonstrates how a primer pair patch can then be seeded with target nucleic acids (e.g., genomic DNA), leading to a target template or target extension product that can be amplified by the pair of specific primers forming a cluster containing the selected genomic region. This embodiment can be used on target nucleic acids (e.g., genomic DNA) without any need to add adaptor sequences. In other words, sample preparation of the target nucleic acids just involves extraction of nucleic acids from a sample of interest, which is then hybridized to the support.

The plurality of primers can comprise the same sequence or different sequences. By way of example, the plurality of primers comprises first and second subsets of primers, the sequence of the primers in the first subset being different from the sequence of the primers in the second subset. If the target nucleic acids comprise a 5′ adaptor sequence, then the first subset can comprise a sequence complementary to a portion of the oligonucleotides on the beads and the second subset of primers can comprise a sequence complementary to the 5′ adaptor sequence.

As used throughout, primers, primer oligonucleotides and amplification oligonucleotides are used interchangeably and are oligonucleotide sequences that are capable of annealing specifically to a polynucleotide sequence to be amplified under conditions encountered in a primer annealing step of an amplification reaction. Generally, the terms nucleic acid, polynucleotide and oligonucleotide are used interchangeably herein. The different terms are not intended to denote any particular difference in size, sequence, or other property unless specifically indicated otherwise. For clarity of description the terms may be used to distinguish one species of molecule from another when describing a particular method or composition that includes several molecular species.

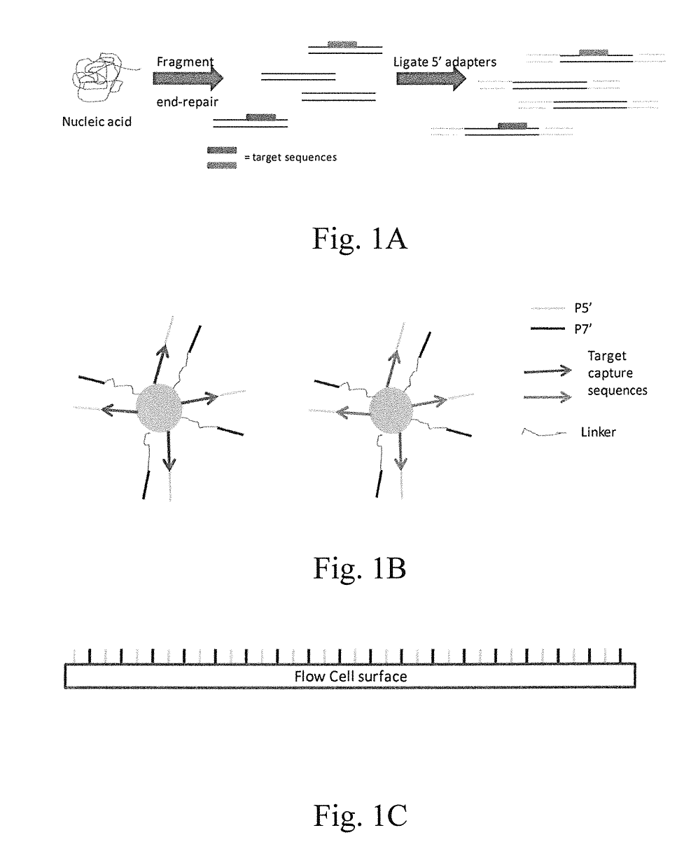

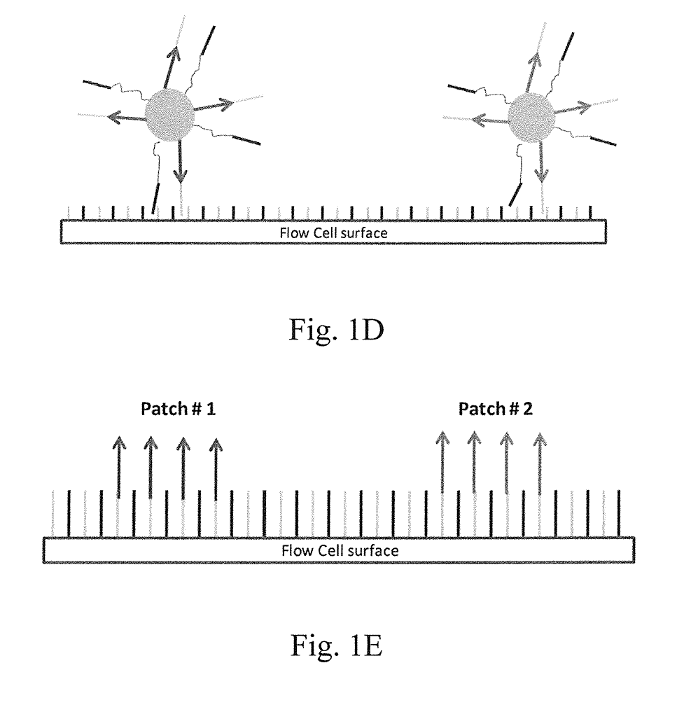

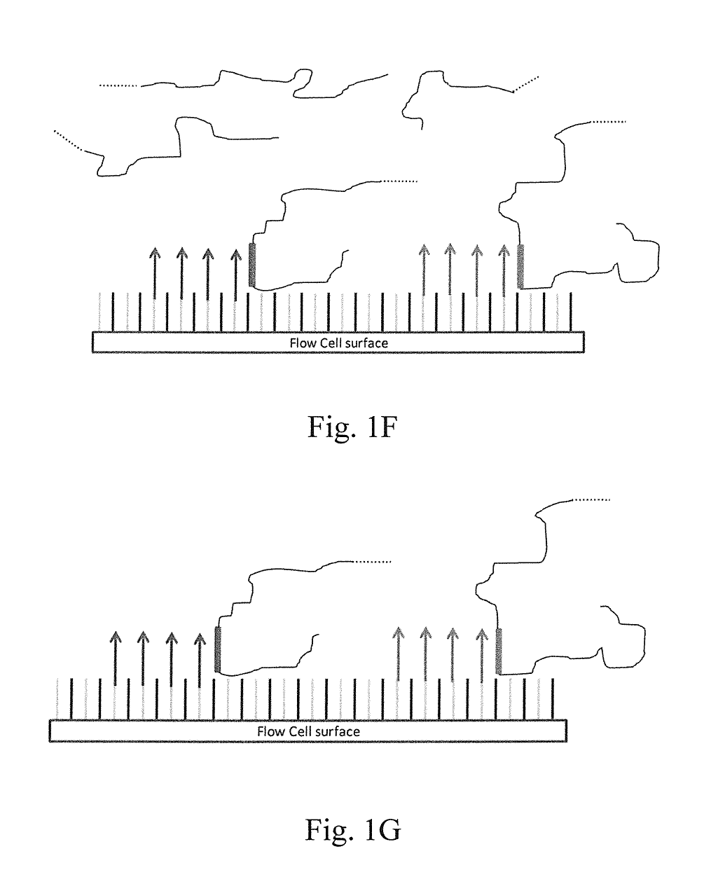

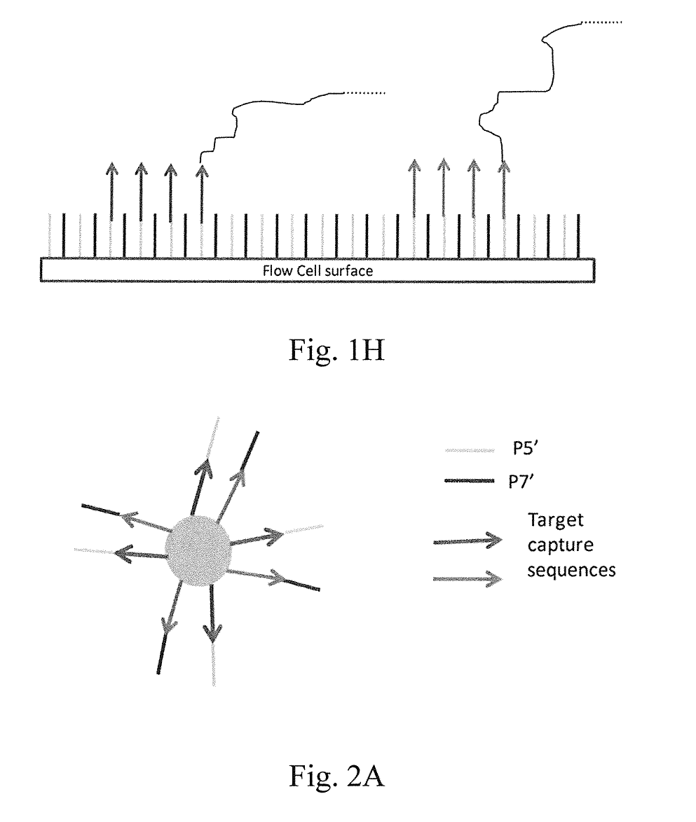

A plurality of oligonucleotides used in the methods set forth herein can include species that function as capture oligonucleotides. The capture oligonucleotides may include a target specific portion, i.e., a sequence of nucleotides capable of annealing to a selected region of a target nucleic acid in a sample. The capture oligonucleotides may comprise a sequence that is specific for a subset of the target nucleic acids in a sample. Thus, only a subset of the nucleic acids in the sample may be selected by the capture oligonucleotides. The capture oligonucleotides may comprise a single species of oligonucleotide, or may comprise two or more species with a different sequence. Thus, the capture oligonucleotide may be two or more sequences, 10 or more sequences, 100 or more sequences, 1000 or more sequences or 10000 or more sequences. The primer binding sequences will generally be of known sequence and will therefore be complementary to a region of known sequence of the oligonucleotide(s) on the bead(s). The capture oligonucleotides may include a capture oligonucleotide and an amplification oligonucleotide or primer. For example, as shown in FIG. 1E, a capture oligonucleotide may be of greater length than amplification oligonucleotides that are attached to the same substrate, in which case the 5′ end of the capture oligonucleotides may comprise a region with the same sequence as one of the amplification oligonucleotides. A portion of the target nucleic acids may be complementary to the 3′ of the capture oligonucleotides (FIG. 1F). The target nucleic acid may contain a region that comprises a sequence identical to one of the amplification oligonucleotides such that upon copying the target nucleic acid, the copy can hybridise to the immobilised amplification oligonucleotide (FIG. 1H). Thus, an oligonucleotide species that is useful in the methods set forth herein can have a capture oligonucleotide, an amplification oligonucleotide or both. Conversely, an oligonucleotide species can lack a capture oligonucleotide, an amplification oligonucleotide or both. In this way the hybridization specificity of an oligonucleotide species can be tailored for a particular application of the methods.

In the provided methods, suitable conditions are applied for extension reactions. The amplification oligonucleotide or primer is extended by sequential addition of nucleotides to generate a capture oligonucleotide complementary to oligonucleotide(s) on the bead(s). Similarly, capture oligonucleotides are extended by sequential addition of nucleotides to generate a target extension product containing sequences complementary to a target nucleic acid. Suitable conditions such as extension buffers/solutions comprising an enzyme with polymerase activity are well known (See, e.g., Molecular Cloning: A Laboratory Manual, (Third Edition), Ed. Sambrook and Russel (2001), which is incorporated by reference herein in its entirety). Examples of enzymes with polymerase activity which can be used in the present invention are DNA polymerase (Klenow fragment, T4 DNA polymerase), heat-stable DNA polymerases from a variety of thermostable bacteria (such as Taq, VENT, Pfu, or Tfl DNA polymerases) as well as their genetically modified derivatives (TaqGold, VENTexo, or Pfu exo). A combination of RNA polymerase and reverse transcriptase can also be used to generate the extension products. Optionally, the enzyme may have strand displacement activity. The nucleoside triphosphate molecules used are typically deoxyribonucleotide triphosphates, for example dATP, dTTP, dCTP, dGTP, or are ribonucleoside triphosphates for example ATP, UTP, CTP, GTP. The nucleoside triphosphate molecules may be naturally or non-naturally occurring.

Optionally, the target nucleic acids are provided by fragmenting nucleic acids (e.g., a genome) to produce the target nucleic acids. Thus, the method can further comprise fragmenting a genome to produce the target nucleic acids. Optionally, the method further comprises adding an adaptor to the 5′ and/or 3′ end of the target nucleic acid fragments. Optionally, the 5′ adaptor and/or 3′ adaptor is the same for each nucleic acid fragment. Adaptor sequences can be added to the 5′ end and/or 3′ end of target nucleic acids. Thus, the target nucleic acids can comprise a 3′ adaptor sequence. Optionally, the 5′ and 3′ adaptor sequence is the same sequence. Optionally, the 5′ adaptor sequence and/or the 3′ adaptor sequence comprises a tag sequence. Optionally, the adaptor comprises a sequence complementary to a primer on the support. Optionally, the target nucleic acids do not comprise an adaptor sequence and an adaptor is ligated to the end of the target extension products prior to amplification of the target extension products.

In the provided methods, a plurality of nucleic acid samples can be provided, tagged to associate each nucleic acid with a specific sample and then pooled prior to contacting the plurality of nucleic acid samples with the support. By way of example, the method can further comprise providing two or more pluralities of nucleic acids each plurality of nucleic acids being from a different source and comprising a tag sequence to identify each nucleic acid as belonging to a particular plurality from a particular source. The two pluralities of nucleic acids can be provided by fragmenting genomic nucleic acid from one source to produce first nucleic acid fragments, ligating adaptors to the 5′ and/or 3′ ends of the first nucleic acid fragments, the adaptors comprising a first sample specific tag sequence, fragmenting a genomic nucleic acid sample from a second source to produce second nucleic acid fragments, ligating adaptors to the 5′ and/or 3′ ends of the second nucleic acid fragments, the adaptors comprising a second sample specific tag sequence and pooling the first and second nucleic acid fragments to provide the two pluralities of nucleic acids. In this way, as many pluralities of nucleic acids as desired and for which a specific tag sequence can be attached can be provided.

Methods for providing target nucleic acids to include 5′- and 3′-adapters include a variety of standard techniques available and known. Exemplary methods of polynucleotide molecule preparation include, but are not limited to, those described in Bentley et al., Nature 456:49-51 (2008); WO 2008/023179; U.S. Pat. No. 7,115,400; and U.S. Patent Application Publication Nos. 2007/0128624; 2009/0226975; 2005/0100900; 2005/0059048; 2007/0110638; and 2007/0128624, each of which is herein incorporated by reference in its entirety. Target nucleic acids are modified to comprise one or more regions of known sequence (e.g., an adapter) located on the 5′ and 3′ ends. The adapters can be linear and can be double- or single-stranded. Optionally, the adapter comprises the indexing tag. When the target nucleic acid molecules comprise known sequences on the 5′ and 3′ ends, the known sequences can be the same or different sequences. Optionally, a known sequence located on the 5′ and/or 3′ ends of the polynucleotide molecules is capable of hybridizing to one or more oligonucleotides immobilized on a surface. For example, a polynucleotide molecule comprising a 5′ known sequence may hybridize to a first plurality of oligonucleotides while the 3′ known sequence may hybridize to a second plurality of oligonucleotides.

Optionally, the target nucleic acids (e.g., a genome) can be fragmented and adaptors can be added to the 5′ and 3′ ends using tagmentation or transposition as described in U.S. Publication No. 2010/0120098, which is incorporated by reference herein in its entirety. Briefly, a “transposition reaction” is a reaction wherein one or more transposons are inserted into target nucleic acids at random sites or almost random sites. Essential components in a transposition reaction are a transposase and DNA oligonucleotides that exhibit the nucleotide sequences of a transposon, including the transferred transposon sequence and its complement (i.e., the non-transferred transposon end sequence) as well as other components needed to form a functional transposition or transposome complex. The DNA oligonucleotides can further comprise additional sequences (e.g., adaptor or primer sequences) as needed or desired. Exemplary transposition complexes, suitable for use in the methods provided herein, include, but are not limited to, those formed by a hyperactive Tn5 transposase and a Tn5-type transposon end or by a MuA transposase and a Mu transposon end comprising R1 and R2 end sequences (See e.g., Goryshin, I. and Reznikoff, W. S., J. Biol. Chem., 273: 7367, 1998; and Mizuuchi, K., Cell, 35: 785, 1983; Savilahti, H, et al., EMBO J., 14: 4893, 1995; which are incorporated by reference herein in their entireties). However, any transposition system that is capable of inserting a transposon end in a random or in an almost random manner with sufficient efficiency to tag target nucleic acids for its intended purpose can be used in the provided methods. Other examples of known transposition systems that could be used in the provided methods include but are not limited to Staphylococcus aureus Tn552, Ty1, Transposon Tn7, Tn/O and IS10, Mariner transposase, Tc1, P Element, Tn3, bacterial insertion sequences, retroviruses, and retrotransposon of yeast (See, e.g., Colegio O R et al., J. Bacteriol., 183: 2384-8, 2001; Kirby C et al., Mol. Microbial., 43: 173-86, 2002; Devine S E, and Boeke J D., Nucleic Acids Res., 22: 3765-72, 1994; International Patent Application No. WO 95/23875; Craig, N L, Science. 271: 1512, 1996; Craig, N L, Review in: Curr Top Microbiol Immunol., 204: 27-48, 1996; Kleckner N, et al., Curr Top Microbiol Immunol., 204: 49-82, 1996; Lampe D J, et al., EMBO J., 15: 5470-9, 1996; Plasterk R H, Curr Top Microbiol Immunol, 204: 125-43, 1996; Gloor, G B, Methods Mol. Biol., 260: 97-114, 2004; Ichikawa H, and Ohtsubo E., J Biol. Chem. 265: 18829-32, 1990; Ohtsubo, F and Sekine, Y, Curr. Top. Microbiol. Immunol. 204: 1-26, 1996; Brown P O, et al., Proc Natl Acad Sci USA, 86: 2525-9, 1989; Boeke J D and Corces V G, Annu Rev Microbiol. 43: 403-34, 1989; which are incorporated herein by reference in their entireties).

The adapters that are added to the 5′ and/or 3′ end of a nucleic acid can comprise a universal sequence. A universal sequence is a region of nucleotide sequence that is common to, i.e., shared by, two or more nucleic acid molecules. Optionally, the two or more nucleic acid molecules also have regions of sequence differences. Thus, for example, the 5′ adapters can comprise identical or universal nucleic acid sequences and the 3′ adapters can comprise identical or universal sequences. A universal sequence that may be present in different members of a plurality of nucleic acid molecules can allow the replication or amplification of multiple different sequences using a single universal primer that is complementary to the universal sequence. Similarly, at least one, two (e.g., a pair) or more universal sequences that may be present in different members of a collection of nucleic acid molecules can allow the replication or amplification of multiple different sequences using at least one, two (e.g., a pair) or more single universal primers that are complementary to the universal sequences. Thus, a universal primer includes a sequence that can hybridize specifically to such a universal sequence. The target nucleic acid sequence-bearing molecules may be modified to attach universal adapters (e.g., non-target nucleic acid sequences) to one or both ends of the different target nucleic acid sequences, the adapters providing sites for hybridization of universal primers. This approach has the advantage that it is not necessary to design a specific pair of primers for each template to be generated, amplified, sequenced, and/or otherwise analyzed; a single pair of primers can be used for amplification of different templates provided that each template is modified by addition of the same universal primer-binding sequences to its 5′ and 3′ ends.

The target nucleic acid molecules can be modified to include any nucleic acid sequence desirable using standard, known methods. Such additional sequences may include, for example, restriction enzyme sites, or oligonucleotide indexing tag in order to permit identification of amplification products of a given nucleic acid sequence. As described herein, the indexing tag can be added to a polynucleotide molecule by inclusion on an adapter or on a primer. Optionally, the indexing tag can be directly ligated to the ends of a polynucleotide molecule.

As used throughout, oligonucleotides or polynucleotide molecules include deoxyribonucleic acids (DNA), ribonucleic acids (RNA) or other form of nucleic acid. The polynucleotide molecule can be any form of natural, synthetic or modified DNA, including, but not limited to, genomic DNA, copy DNA, complementary DNA, or recombinant DNA. Alternatively, the polynucleotide molecule can be any form of natural, synthetic or modified RNA, including, but not limited to mRNA, ribosomal RNA, microRNA, siRNA or small nucleolar RNA. The polynucleotide molecule can be partially or completely in double-stranded or single-stranded form. The terms “nucleic acid,” “nucleic acid molecule,” “oligonucleotide,” and “polynucleotide” are used interchangeably throughout. The different terms are not intended to denote any particular difference in size, sequence, or other property unless specifically indicated otherwise. For clarity of description the terms may be used to distinguish one species of molecule from another when describing a particular method or composition that includes several molecular species.

As used throughout, the term “target nucleic acid” can be any molecule to be selected and, optionally, amplified or sequenced. Target nucleic acids for use in the provided methods may be obtained from any biological sample using known, routine methods. Suitable biological samples include, but are not limited to, a blood sample, biopsy specimen, tissue explant, organ culture, biological fluid or any other tissue or cell preparation, or fraction or derivative thereof or isolated therefrom. The biological sample can be a primary cell culture or culture adapted cell line including but not limited to genetically engineered cell lines that may contain chromosomally integrated or episomal recombinant nucleic acid sequences, immortalized or immortalizable cell lines, somatic cell hybrid cell lines, differentiated or differentiatable cell lines, transformed cell lines, stem cells, germ cells (e.g. sperm, oocytes), transformed cell lines and the like. For example, polynucleotide molecules may be obtained from primary cells, cell lines, freshly isolated cells or tissues, frozen cells or tissues, paraffin embedded cells or tissues, fixed cells or tissues, and/or laser dissected cells or tissues. Biological samples can be obtained from any subject or biological source including, for example, human or non-human animals, including mammals and non-mammals, vertebrates and invertebrates, and may also be any multicellular organism or single-celled organism such as a eukaryotic (including plants and algae) or prokaryotic organism, archaeon, microorganisms (e.g. bacteria, archaea, fungi, protists, viruses), and aquatic plankton.

The target nucleic acid described herein can be of any length suitable for use in the provided methods. For example, the target nucleic acids can be at least 10, at least 20, at least 30, at least 40, at least 50, at least 75, at least 100, at least 150, at least 200, at least 250, at least 500, or at least 1000 nucleotides in length or longer. Generally, if the target nucleic acid is a small RNA molecule, the target nucleic acid will be at least 10 nucleotides in length. Thus, the target nucleic acid sequences can comprise RNA molecules, for example, small RNA molecules including, but not limited to miRNA molecules, siRNA molecules, tRNA molecules, rRNA molecules, and combinations thereof. In some embodiments, the target nucleic acid sequence comprises single-stranded DNA.

The term immobilized as used herein is intended to encompass direct or indirect attachment to a solid support via covalent or non-covalent bond(s). In certain embodiments of the invention, covalent attachment may be used, but generally all that is required is that the molecules (for example, nucleic acids) remain immobilised or attached to a support under conditions in which it is intended to use the support, for example in applications requiring nucleic acid amplification and/or sequencing. Typically oligonucleotides to be used as capture oligonucleotides or amplification oligonucleotides are immobilized such that a 3′ end is available for enzymatic extension and at least a portion of the sequence is capable of hybridizing to a complementary sequence. Immobilization can occur via hybridization to a surface attached oligonucleotide, in which case the immobilised oligonucleotide or polynucleotide may be in the 3′-5′ orientation. Alternatively, immobilization can occur by means other than base-pairing hybridization, such as the covalent attachment set forth above.

The term support as used herein refers to any substrate or matrix to which molecules can be attached, such as for example latex beads, dextran beads, polystyrene surfaces, polypropylene surfaces, polyacrylamide gel, gold surfaces, glass surfaces and silicon wafers. The solid support may be a planar glass surface. The solid support may be mounted on the interior of a flow cell to allow the interaction with solutions of various reagents.

Optionally, the support may comprise an inert substrate or matrix which has been functionalised, for example, by the application of a layer or coating of an intermediate material comprising reactive groups that permit covalent attachment to molecules such as polynucleotides. By way of non-limiting example such supports may include polyacrylamide hydrogel layers on an inert substrate such as glass. In such embodiments the molecules (for example, polynucleotides) may be directly covalently attached to the intermediate layer (for example, a hydrogel) but the intermediate layer may itself be non-covalently attached to other layers of the substrate or matrix (for example, a glass substrate). Covalent attachment to a solid support is to be interpreted accordingly as encompassing this type of arrangement.

The creation of patterned surfaces, for example, has been described in European Patent No. 2291533, which is incorporated by reference herein in its entirety. The creation of these oligo patches ensures the presence of a vast amount of oligonucleotides on the surface that can efficiently capture the target sequences.

Once the target extension products are generated, the target extension products or captured target nucleic acids can be amplified. Optionally, the amplification comprises using the primers on the support. Alternatively, the amplification can comprise using one primer in solution and one primer on the support. In some embodiments, amplification produces clusters of amplified target nucleic acid molecules. Generally amplification reactions use at least two amplification oligonucleotides, often denoted ‘forward’ and ‘reverse’ primers. Generally amplification oligonucleotides are single stranded polynucleotide structures. They may also contain a mixture of natural or non-natural bases and also natural and non-natural backbone linkages, provided, at least in some embodiments, that any non-natural modifications do not permanently or irreversibly preclude function as a primer—that being defined as the ability to anneal to a template polynucleotide strand during conditions of an extension or amplification reaction and to act as an initiation point for the synthesis of a new polynucleotide strand complementary to the annealed template strand. Primers may additionally comprise non-nucleotide chemical modifications, for example to facilitate covalent attachment of the primer to a support. Certain chemical modifications may themselves improve the function of the molecule as a primer or may provide some other useful functionality, such as providing a cleavage site that enables the primer (or an extended polynucleotide strand derived therefrom) to be cleaved from a support.

Nucleic acid amplification includes the process of amplifying or increasing the numbers of a nucleic acid template and/or of a complement thereof that are present, by producing one or more copies of the template and/or or its complement. Amplification can be carried out by a variety of known methods under conditions including, but not limited to, thermocycling amplification or isotheraml amplification. For example, methods for carrying out amplification are described in U.S. Publication No. 2009/0226975; WO 98/44151; WO 00/18957; WO 02/46456; WO 06/064199; and WO 07/010251; which are incorporated by reference herein in their entireties. Thus, amplification can occur on the surface to which the nucleic acid molecules are attached. This type of amplification can be referred to as solid phase amplification, which when used in reference to nucleic acids, refers to any nucleic acid amplification reaction carried out on or in association with a surface (e.g., a support). For example, all or a portion of the amplified products are synthesized by extension of an immobilized primer. Solid phase amplification reactions are analogous to standard solution phase amplifications except that at least one of the amplification oligonucleotides is immobilized on a surface (e.g., a solid support).

Solid-phase amplification may comprise a nucleic acid amplification reaction comprising only one species of oligonucleotide primer immobilized to a surface. Alternatively, the surface may comprise a plurality of first and second different immobilized oligonucleotide primer species. Solid-phase amplification may comprise a nucleic acid amplification reaction comprising one species of oligonucleotide primer immobilized on a solid surface and a second different oligonucoleotide primer species in solution. Solid phase nucleic acid amplification reactions generally comprise at least one of two different types of nucleic acid amplification, interfacial and surface (or bridge) amplification. For instance, in interfacial amplification, the solid support comprises a template nucleic acid molecule that is indirectly immobilized to the solid support by hybridization to an immobilized oligonucleotide primer, the immobilized primer may be extended in the course of a polymerase-catalyzed, template-directed elongation reaction (e.g., primer extension) to generate an immobilized polynucleotide molecule that remains attached to the solid support. After the extension phase, the nucleic acids (e.g., template and its complementary product) are denatured such that the template nucleic acid molecule is released into solution and made available for hybridization to another immobilized oligonucleotide primer. The template nucleic acid molecule may be made available in 1, 2, 3, 4, 5 or more rounds of primer extension or may be washed out of the reaction after 1, 2, 3, 4, 5 or more rounds of primer extension.

In surface (or bridge) amplification, an immobilized nucleic acid molecule hybridizes to an immobilized oligonucleotide primer. The 3′ end of the immobilized nucleic acid molecule provides the template for a polymerase-catalyzed, template-directed elongation reaction (e.g., primer extension) extending from the immobilized oligonucleotide primer. The resulting double-stranded product “bridges” the two primers and both strands are covalently attached to the support. In the next cycle, following denaturation that yields a pair of single strands (the immobilized template and the extended-primer product) immobilized to the solid support, both immobilized strands can serve as templates for new primer extension.

Optionally, amplification of the adapter-target-adapters or library of nucleic acid sequences results in clustered arrays of nucleic acid colonies, analogous to those described in U.S. Pat. No. 7,115,400; U.S. Publication No. 2005/0100900; WO 00/18957; and WO 98/44151, which are incorporated by reference herein in their entireties. Clusters and colonies are used interchangeably and refer to a plurality of copies of a nucleic acid sequence and/or complements thereof attached to a surface. Typically, the cluster comprises a plurality of copies of a nucleic acid sequence and/or complements thereof, attached via their 5′ termini to the surface. The copies of nucleic acid sequences making up the clusters may be in a single or double stranded form.

Clusters may be detected, for example, using a suitable imaging means, such as, a confocal imaging device or a charge coupled device (CCD) camera. Exemplary imaging devices include, but are not limited to, those described in U.S. Pat. Nos. 7,329,860; 5,754,291; and 5,981,956; and WO 2007/123744, each of which is herein incorporated by reference in its entirety. The imaging means may be used to determine a reference position in a cluster or in a plurality of clusters on the surface, such as the location, boundary, diameter, area, shape, overlap and/or center of one or a plurality of clusters (and/or of a detectable signal originating therefrom). Such a reference position may be recorded, documented, annotated, converted into an interpretable signal, or the like, to yield meaningful information. The signal may, for instance, take the form of a detectable optical signal emanating from a defined and identifiable location, such as a fluorescent signal, or may be a detectable signal originating from any other detectable label as provided herein. The reference position of a signal generated from two or more clusters may be used to determine the actual physical position on the surface of two clusters that are related by way of being the sites for simultaneous sequence reads from different portions of a common target nucleic acid.

Following amplification, the amplified target extension products or target nucleic acids can be sequenced. Thus, for example, the provided methods can further comprising sequencing the amplified target nucleic acids. Optionally, the sequencing comprises sequencing-by-synthesis or sequencing-by-ligation.

Sequencing by synthesis, for example, is a technique wherein nucleotides are added successively to a free 3′ hydroxyl group, typically provided by annealing of an oligonucleotide primer (e.g., a sequencing primer), resulting in synthesis of a nucleic acid chain in the 5′ to 3′ direction. These and other sequencing reactions may be conducted on the herein described surfaces bearing nucleic acid clusters. The reactions comprise one or a plurality of sequencing steps, each step comprising determining the nucleotide incorporated into a nucleic acid chain and identifying the position of the incorporated nucleotide on the surface. The nucleotides incorporated into the nucleic acid chain may be described as sequencing nucleotides and may comprise one or more detectable labels. Suitable detectable labels, include, but are not limited to, haptens, radionucleotides, enzymes, fluorescent labels, chemiluminescent labels, and/or chromogenic agents. One method for detecting fluorescently labeled nucleotides comprises using laser light of a wavelength specific for the labeled nucleotides, or the use of other suitable sources of illumination. The fluorescence from the label on the nucleotide may be detected by a CCD camera or other suitable detection means. Suitable instrumentation for recording images of clustered arrays is described in WO 07/123744, the contents of which are incorporated herein by reference herein in its entirety.

Optionally, cycle sequencing is accomplished by stepwise addition of reversible terminator nucleotides containing, for example, a cleavable or photobleachable dye label as described, for example, in U.S. Pat. No. 7,427,673; U.S. Pat. No. 7,414,116; WO 04/018497; WO 91/06678; WO 07/123744; and U.S. Pat. No. 7,057,026, the disclosures of which are incorporated herein by reference in their entireties. The availability of fluorescently-labeled terminators in which both the termination can be reversed and the fluorescent label cleaved facilitates efficient cyclic reversible termination (CRT) sequencing. Polymerases can also be co-engineered to efficiently incorporate and extend from these modified nucleotides.

Alternatively, pyrosequencing techniques may be employed. Pyrosequencing detects the release of inorganic pyrophosphate (PPi) as particular nucleotides are incorporated into the nascent strand (Ronaghi et al., (1996) “Real-time DNA sequencing using detection of pyrophosphate release.” Analytical Biochemistry 242(1), 84-9; Ronaghi, M. (2001) “Pyrosequencing sheds light on DNA sequencing.” Genome Res. 11(1), 3-11; Ronaghi, M., Uhlen, M. and Nyren, P. (1998) “A sequencing method based on real-time pyrophosphate.” Science 281(5375), 363; U.S. Pat. No. 6,210,891; U.S. Pat. No. 6,258,568; and U.S. Pat. No. 6,274,320, the disclosures of which are incorporated herein by reference in their entireties). In pyrosequencing, released PPi can be detected by being immediately converted to adenosine triphosphate (ATP) by ATP sulfurylase, and the level of ATP generated is detected via luciferase-produced photons.

Additional exemplary sequencing-by-synthesis methods that can be used with the methods described herein include those described in U.S. Patent Publication Nos. 2007/0166705; 2006/0188901; 2006/0240439; 2006/0281109; 2005/0100900; U.S. Pat. No. 7,057,026; WO 05/065814; WO 06/064199; WO 07/010251, the disclosures of which are incorporated herein by reference in their entireties.

Alternatively, sequencing by ligation techniques are used. Such techniques use DNA ligase to incorporate oligonucleotides and identify the incorporation of such oligonucleotides and are described in U.S. Pat. No. 6,969,488; U.S. Pat. No. 6,172,218; and U.S. Pat. No. 6,306,597; the disclosures of which are incorporated herein by reference in their entireties. Other suitable alternative techniques include, for example, fluorescent in situ sequencing (FISSEQ), and Massively Parallel Signature Sequencing (MPSS).

Disclosed are materials, compositions, and components that can be used for, can be used in conjunction with, can be used in preparation for, or are products of the disclosed methods and compositions. These and other materials are disclosed herein, and it is understood that when combinations, subsets, interactions, groups, etc. of these materials are disclosed that while specific reference of each various individual and collective combinations and permutation may not be explicitly disclosed, each is specifically contemplated and described herein. For example, if a method is disclosed and discussed and a number of modifications that can be made to the method steps are discussed, each and every combination and permutation of the method steps, and the modifications that are possible are specifically contemplated unless specifically indicated to the contrary. Likewise, any subset or combination of these is also specifically contemplated and disclosed. This concept applies to all aspects of this disclosure. Thus, if there are a variety of additional steps that can be performed it is understood that each of these additional steps can be performed with any specific method steps or combination of method steps of the disclosed methods, and that each such combination or subset of combinations is specifically contemplated and should be considered disclosed.

Throughout this application, various publications are referenced. The disclosures of these publications in their entireties are hereby incorporated by reference into this application.

A number of embodiments have been described. Nevertheless, it will be understood that various modifications may be made. Accordingly, other embodiments are within the scope of the claims.

1. A method of selecting and amplifying target nucleic acids on a support, the method comprising:
(a) providing a plurality of beads each bead comprising one or more oligonucleotides;
(b) providing a support with a plurality of primers with a sequence complementary to at least a portion of the oligonucleotides on the beads;
(c) contacting the beads with the support under conditions wherein the oligonucleotides on the beads bind to the primers on the support; and wherein each bead comprises one or more pairs of oligonucleotides comprising forward and reverse primer sequences specific to a target nucleic acid sequence;
(d) performing an extension reaction by extending the primers on the support to produce extension products comprising a sequence complementary to at least a portion of the oligonucleotides, wherein the extension products are capture oligonucleotides;
(e) contacting the support comprising the capture oligonucleotides with the target nucleic acids, the target nucleic acids potentially comprising sequences complementary to one or more of the capture oligonucleotides;
(f) extending the capture oligonucleotides bound to target nucleic acids to produce target extension products comprising a sequence complementary to at least a portion of the target nucleic acids; and
(g) amplifying the target extension products.

2. The method of claim 1, wherein each bead comprises a different oligonucleotide sequence.

3. The method of claim 1, wherein the plurality of beads comprises two or more subsets of beads.

4-7. (canceled)

8. The method of claim 1, wherein the plurality of primers comprises first and second subsets of primers, the sequence of the primers in the first subset being different from the sequence of the primers in the second subset.

9. The method of claim 1, wherein the target nucleic acids comprise a 5′ adaptor sequence.

10. The method of claim 9, wherein the first subset comprises a sequence complementary to a portion of the oligonucleotides on the beads and the second subset of primers comprises a sequence complementary to the 5′ adaptor sequence.

11. The method of claim 1, wherein the target nucleic acids comprise a 3′ adaptor sequence.

12. The method of claim 11, wherein the 5′ and 3′ adaptor sequence is the same sequence.

13. The method of claim 11, wherein the 5′ adaptor sequence and/or the 3′ adaptor sequence comprises a tag sequence.

14. (canceled)

15. The method of claim 1, wherein the capture oligonucleotides comprise the forward and reverse primer sequences and wherein the target nucleic acid binds the forward primer sequence.

16. The method of claim 15, wherein the target extension products are amplified using the forward and reverse primer sequences in the capture oligonucleotides.

17-19. (canceled)

20. The method of claim 1, wherein the amplification comprises using the primers on the support.

21. The method of claim 1, wherein the amplification comprises using one primer in solution and one primer on the support.

22. The method of claim 1, further comprising fragmenting a genome to produce the target nucleic acids.

23. The method of claim 22, further comprising adding an adaptor to the 5′ and/or 3′ end of the target nucleic acid fragments.

24. The method of claim 23, wherein the 5′ adaptor sequence is the same for each nucleic acid fragment.

25. The method of claim 23, wherein the 3′ adaptor sequence is the same for each nucleic acid fragment.

26. (canceled)

27. The method of claim 1, further comprising sequencing the amplified target nucleic acids.

28-29. (canceled)

30. The method of claim 1, further comprising providing two or more pluralities of nucleic acids each plurality of nucleic acids being from a different source and comprising a tag sequence to identify each nucleic acid as belonging to a particular plurality from a particular source.

31. The method of claim 30, wherein two pluralities of nucleic acids are provided by fragmenting a nucleic acid sample from one source to produce first nucleic acid fragments, adding adaptors to the 5′ and/or 3′ ends of the first nucleic acid fragments, the adaptors comprising a first sample specific tag sequence, fragmenting a nucleic acid sample from a second source to produce second nucleic acid fragments, adding adaptors to the 5′ and/or 3′ ends of the second nucleic acid fragments, the adaptors comprising a second sample specific tag sequence, and pooling the first and second nucleic acid fragments to provide the two pluralities of nucleic acids.
